(19) United States
(12) Patent Application Publication
(10) Pub. No.: US 2024/0058550 A1
(43) Pub. Date: Feb. 22, 2024

(54) NEBULIZER

(71) Applicant: OMRON HEALTHCARE CO., LTD., Muko-shi (JP)

(72) Inventors: Hidetaka TOGO, Kyoto (JP); Hiroko YOSHINO, Kyoto (JP); Toshifumi MATSUMOTO, Kyoto (JP)

(21) Appl. No.: 18/385,965

(22) Filed: Nov. 1, 2023

Related U.S. Application Data
(63) Continuation of application No. PCT/JP2022/022328, filed on Jun. 1, 2022.

(30) Foreign Application Priority Data
Jun. 25, 2021 (JP) 2021-105971

(51) Int. Cl. A61M 11/00; B05B 17/06

(52) U.S. Cl. CPC A61M 11/00; B05B 17/0607

(57) ABSTRACT

In a nebulizer of the present invention, a main body is being mounted with a power supply unit and an oscillation unit generating an oscillation output including a first frequency component and a second frequency component different from each other. A first replacement member is being mounted with an atomization unit configured to atomize, using the first frequency component, a first liquid that is supplied. A second replacement member is being mounted with an atomization unit configured to atomize, using the second frequency component, a second liquid that is supplied. The first and/or the second replacement members include a functional unit configured to operate with an additional frequency component different from the first and second frequency components. A replacement member attached to the main body receives the oscillation output including the first, second and additional frequency components from the main body.

### CROSS-REFERENCE TO RELATED APPLICATIONS

This is a continuation application of International Application No. PCT/JP2022/022328, with an International filing date of Jun. 1, 2022, which claims priority of Japanese Patent Application No. 2021-105971 filed on Jun. 25, 2021, the entire content of which is hereby incorporated by reference.

### TECHNICAL FIELD

The present invention relates to a nebulizer, and more specifically to a nebulizer that atomizes and ejects a liquid such as a chemical liquid.

### BACKGROUND ART

Conventionally, as this type of nebulizer, a nebulizer including a main body being mounted with one ultrasonic vibrator and a replacement member including a sheet having a mesh portion is known as disclosed in Patent Document 1 (JP-A-2018-050821), for example. In a state where the replacement member is attached to the main body, the mesh portion of the sheet is supported facing a vibration surface of the ultrasonic vibrator in a slightly inclined state. During operation, in a state where a chemical liquid is supplied between the vibration surface and the mesh portion, the vibration surface of the ultrasonic vibrator is vibrated at a certain frequency (within a range of 180 kHz±5 kHz) substantially matching the resonance frequency of the ultrasonic vibrator. This causes the chemical liquid to be atomized and sprayed through the mesh portion.

### SUMMARY OF THE INVENTION

Depending on the type (difference in viscosity) or the like of chemical liquid, for example, the frequency (referred to as “target frequency”) suitable for atomization may be greatly different, for example, 100 kHz, 300 kHz, or the like, rather than the vicinity of 180 kHz. However, in the above-described nebulizer, since one ultrasonic vibrator is driven at a certain frequency (within a range of 180 kHz±5 kHz), there is a problem that it is difficult to appropriately atomize chemical liquids having different target frequencies and to respond to various needs.

Therefore, an object of the present invention is to provide a nebulizer that can appropriately atomize liquids having different target frequencies suitable for atomization and can thus respond to various needs.

In order to achieve the object, a nebulizer of the present disclosure is a nebulizer that atomizes and ejects a liquid, the nebulizer comprising:
a main body being mounted with a power supply unit and an oscillation unit, the oscillation unit receiving power supply from the power supply unit and generating an oscillation output including a predetermined first frequency component and a predetermined second frequency component different from each other;
a first replacement member being mounted with an atomization unit configured to atomize, using the first frequency component, a first liquid that is supplied; and
a second replacement member being mounted with an atomization unit configured to atomize, using the second frequency component, a second liquid that is supplied,
wherein
the first replacement member and/or the second replacement member include a functional unit configured to operate with a predetermined additional frequency component different from the first and second frequency components,
the oscillation unit of the main body generates the oscillation output including the additional frequency component in addition to the first and second frequency components, and
the first replacement member or the second replacement member is attached to the main body in a replaceable manner, and a replacement member attached to the main body receives the oscillation output including the first frequency component, the second frequency component, and the additional frequency component from the main body.

In the present specification, for the “power supply unit”, a battery may be used, or a unit that converts a commercial power supply may be used.

The “first liquid” and the “second liquid” refer to, for example, chemical liquids having different viscosities from each other.

The “first frequency component” and the “second frequency component” have frequencies (target frequencies) suitable for atomization of the “first liquid” and the “second liquid”, respectively.

The “first replacement member” and the “second replacement member” typically include ultrasonic vibrators for atomization operations, respectively. The “first replacement member” may further include a liquid supply unit that supplies the first liquid to its own atomization unit, and the “second replacement member” may further include a liquid supply unit that supplies the second liquid to its own atomization unit.

The “functional unit” means an element that operates by receiving the oscillation output from the main body (to this extent, the “functional unit” is a concept including the atomization unit as well). The “functional unit” may be, for example, a liquid supply unit (including an infusion pump, for example) that supplies a liquid to the atomization unit, or an air blower unit (for example, an air blowing fan) that assists ejection of atomized liquid.

The “attached replacement member” refers to the first replacement member or the second replacement member attached to the main body.

The system for transmitting the “oscillation output” from the main body to the first replacement member or the second replacement member may be a wireless power transmission system or a wired power transmission system.

### BRIEF DESCRIPTION OF THE DRAWINGS

FIG. 1 is a perspective view showing an exploded state of a nebulizer according to an embodiment of the present invention.

FIG. 2 is a view schematically showing an internal structure of the nebulizer as viewed from a side.

FIG. 3 is a diagram showing a block configuration of a control system of the nebulizer.

FIG. 4 is a diagram showing frequency components included in an oscillation output by a control unit of the nebulizer.

FIG. 5 is a view showing a usage mode of the nebulizer by a user.

FIG. 6 is a diagram showing a block configuration that may be taken by a modification example of the nebulizer.

FIG. 7A is a diagram showing a schematic operation flow of a control unit in the nebulizer of FIG. 6.

FIG. 7B is a diagram showing a detailed operation flow of an initial setting included in the schematic operation flow.

FIG. 7C is a diagram showing a detailed operation flow of another initial setting included in the schematic operation flow.

FIG. 8A is a diagram showing a change in admittance (or current value) of an atomization unit or a functional unit of each replacement member according to a change in driving frequency.

FIG. 8B is a diagram showing a change in impedance (or voltage value) of an atomization unit or a functional unit of each replacement member according to a change in driving frequency.

### DETAILED DESCRIPTION

Hereinafter, embodiments of the present invention will be described in detail with reference to the drawings.

(Configuration of Nebulizer)

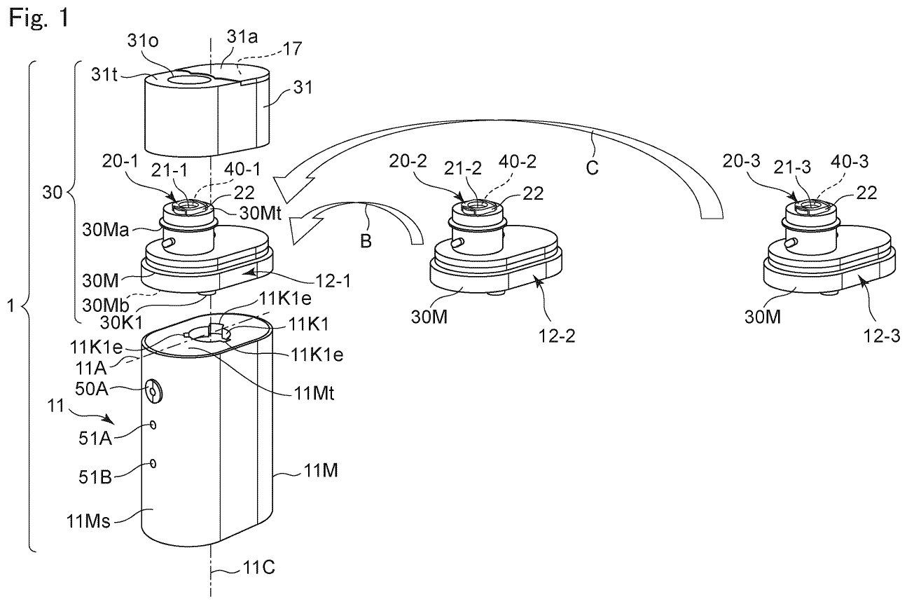

FIG. 1 shows a nebulizer (a whole of which is indicated by reference 1) according to an embodiment of the present invention in an exploded state. The nebulizer 1 mainly includes a main body 11 having a main body casing 11M, a spray unit 12-1 as a first replacement member to be replaced and attached to the main body 11, a spray unit 12-2 as a second replacement member, and a spray unit 12-3 as a third replacement member.

In this example, the main body casing 11M constituting the main body 11 has an oval planar shape (having a long axis 11A extending from the left front to the right back in FIG. 1), and has a columnar outer shape extending in a direction of a longitudinal axis 11C (in this example, a vertical direction). A power switch 50A for turning on and off the power of the nebulizer 1 and display lamps 51A and 51B for indicating an operation state of the nebulizer 1 are provided on a front surface (left front side surface in FIG. 1) 11Ms of the main body casing 11M. A recess 11K1 having a substantially short cylindrical outer shape is provided in a central portion (through which the longitudinal axis 11C passes) of an upper wall 11Mt of the main body casing 11M, as an element for detachably attaching the spray unit 12-1 to the main body 11. In this example, the recess 11K1 includes orientation grooves 11K1e, 11K1e, and 11K1e extending radially outward in portions corresponding to specific orientations (in this example, three orientations at intervals of 120°) around the longitudinal axis 11C.

The spray unit 12-1 includes a base casing 30M having the same oval planar shape as that of the main body casing 11M, and a cover member 31 covering the base casing 30M. The cover member 31 is detachably fitted and attached to the base casing 30M in the direction of the longitudinal axis 11C (in this example, from above). The base casing 30M and the cover member 31 constitute an attachment casing 30.

In this example, the base casing 30M includes an upper stage accommodating portion 30Ma protruding upward in a columnar shape at a portion eccentric to the left front side from the longitudinal axis 11C. The upper stage accommodating portion 30Ma accommodates a horn vibrator 40-1 as a vibration unit suitable for atomizing a first liquid. In this example, a mesh member 20-1 is placed on a top surface 30Mt of the upper stage accommodating portion 30Ma in a state of facing the horn vibrator 40-1. In this example, the mesh member 20-1 includes a sheet 21-1 including a mesh portion suitable for atomizing the first liquid, and a flange portion 22 supporting a peripheral edge of the sheet 21-1. The “mesh portion” means an element that has a plurality of through holes in a sheet (or a plate material) and allows a liquid to pass through these through holes to be atomized. In this example, the mesh member 20-1 is configured to be disposable after one use. In this example, the horn vibrator 40-1 and the mesh member 20-1 constitute an atomization unit.

A projection 30K1 having a substantially short columnar outer shape is provided in a central portion (through which the longitudinal axis 11C passes) of a bottom wall 30Mb of the spray unit 12-1 as an element for detachably attaching the spray unit 12-1 to the main body 11. In this example, the projection 30K1 has a shape corresponding to the recess 11K1 of the main body casing 11M. That is, the projection 30K1 has a substantially cylindrical shape, and includes an enlarged diameter portion (not shown) protruding radially outward at portions corresponding to specific orientations (in this example, three orientations at intervals of 120°) around the longitudinal axis 11C. Thus, when the spray unit 12-1 (base casing 30M) is brought close to the main body 11 (main body casing 11M) in a direction of the longitudinal axis 11C (in this example, from above), the projection 30K1 is fitted to the recess 11K1, and the spray unit 12-1 is easily attached to the main body 11. Once the spray unit 12-1 is attached to the main body 11, the attached state is maintained by the frictional force between the recess 11K1 and the projection 30K1. When the user applies a force exceeding the frictional force to separate the spray unit 12-1 from the main body 11 in the direction of the longitudinal axis 11C, the spray unit 12-1 is easily detached from the main body 11.

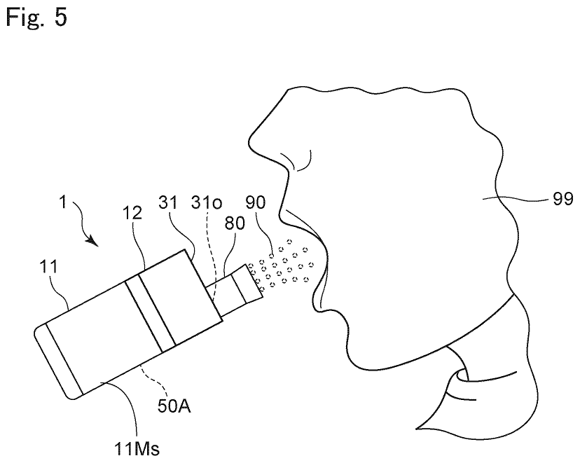

The cover member 31 has the same oval planar shape as that of the base casing 30M and has a cylindrical outer shape extending in a direction of the longitudinal axis 11C. A circular opening 31o is provided in a portion of a top wall 31t of the cover member 31 eccentric to the left front side from the longitudinal axis 11C. In a state where the cover member 31 is attached to the base casing 30M, an edge portion of the opening 31o presses a flange portion 22 of the mesh member 20-1 in a direction of the longitudinal axis 11C (in this example, from above). As a result, the sheet 21-1 including the mesh portion is positioned with respect to the horn vibrator 40-1. For example, as shown in FIG. 5, a mouthpiece 80 as a pipe member is detachably attached to the opening 31o from the outside of the cover member 31.

In addition, the cover member 31 includes a lid portion 31a that can be opened and closed by a hinge and a liquid reservoir 17 as a liquid supply unit provided at a position immediately below the lid portion 31a at a portion corresponding to the right back side from the opening 31o in the top wall 31t. With the cover member 31 attached to the base casing 30M, the user can temporarily open the lid portion 31a and put the first liquid into the liquid reservoir 17 in this example.

The spray units 12-2 and 12-3 have different mesh members 20-2 and 20-3, respectively, instead of the mesh member 20-1, and have different horn vibrators 40-2 and 40-3, respectively, instead of the horn vibrator 40-1. The mesh member 20-2 and the horn vibrator 40-2 of the spray unit 12-2 are suitable for atomizing a second liquid different from the first liquid. The mesh member 20-3 and the horn vibrator 40-3 of the spray unit 12-3 are suitable for atomizing a third liquid different from the first and second liquids. The spray units 12-2 and 12-3 are configured in the same manner as the spray unit 12-1 except for the above. As indicated by arrows B and C in FIG. 1, the spray units 12-2 and 12-3 can be attached to the main body 11 in such a manner as to be replaced with the spray units 12-1, respectively.

The first, second, and third liquids refer to, for example, chemical liquids having different viscosities (various types of chemical liquids for inhalation are commercially available). In this example, it is assumed that the hole diameters of the mesh portions of the mesh members 20-1, 20-2, and 20-3 and the thicknesses of the sheets 21-1, 21-2, and 21-3 are set so that the first, second, and third liquids can be appropriately atomized, respectively. In addition, it is assumed that frequencies (target frequencies) suitable for atomization by the horn vibrators 40-1, 40-2, and 40-3 in the spray units 12-1, 12-2, and 12-3 are f1≈180 kHz, f2≈300 kHz, and f3≈500 kHz, respectively.

Hereinafter, for convenience of description, the spray units 12-1, 12-2, and 12-3 are collectively referred to as a spray unit 12, the sheets 21-1, 21-2, and 21-3 are collectively referred to as a sheet 21, and the horn vibrators 40-1, 40-2, and 40-3 are collectively referred to as a horn vibrator 40, appropriately.

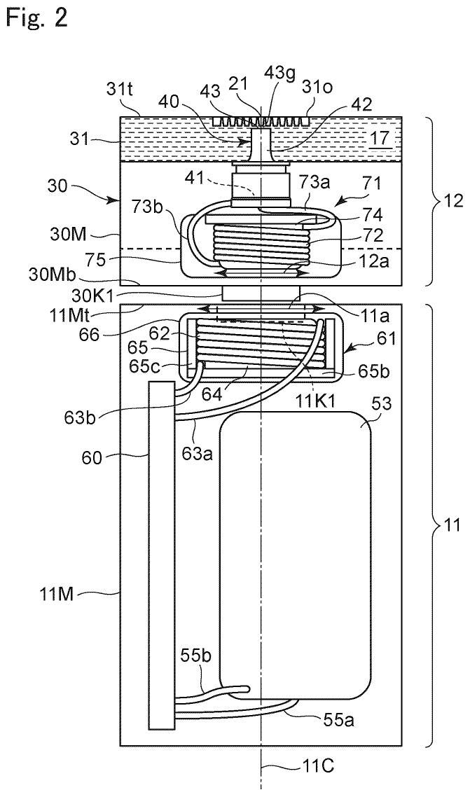

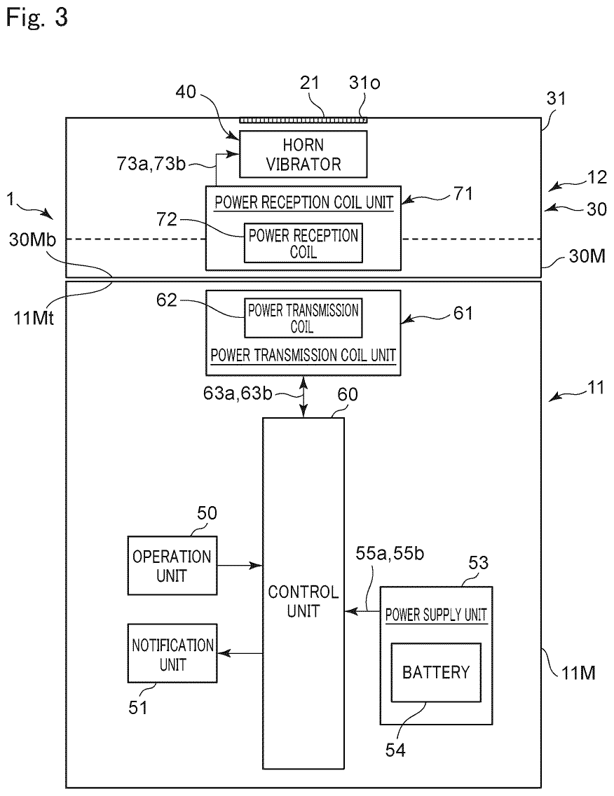

FIG. 2 schematically shows an internal structure of the nebulizer 1 as viewed from a side. FIG. 3 shows a block configuration of a control system of the nebulizer 1. For easy understanding, in FIG. 2, a slight gap for showing the projection 30K1 of the base casing 30M is provided between the base casing 30M of the spray unit 12 and the main body casing 11M. In FIG. 3, a gap between the base casing 30M of the spray unit 12 and the main body casing 11M is not intended.

As can be seen from FIG. 3, the main body 11 is mounted with a control unit 60, an operation unit 50, a notification unit 51, a power supply unit 53, and a power transmission coil unit 61 to accommodate them in the main body casing 11M. In this example, the control unit 60 comprises a printed circuit board (PCB) to control the entire operation of the nebulizer 1. The operation unit 50 includes the power switch 50A described above, with which instructions for turning on and off the power of the nebulizer 1 and various other instructions from the user are input. The power supply unit 53 includes a battery 54 in this example and supplies power to each unit (including the control unit 60) of the nebulizer 1. The control unit 60 and the power supply unit 53 are connected by wirings 55a and 55b. The power supply unit 53 may be a unit obtained by converting a commercial power supply. The notification unit 51 includes the display lamps 51A and 51B described above and a buzzer (not shown), and displays the operating state of the nebulizer 1 and/or generates an alarm display or an alarm sound. For example, the display lamp 51A displays on and off of the power, and the display lamp 51B displays the remaining level of the battery 54.

As shown in FIG. 2, the power transmission coil unit 61 includes, in this example, a pole piece 64 made of a substantially columnar magnetic body, a yoke 65 made of a magnetic body including an end plate portion 65b in contact with a lower end of the pole piece 64 and an outer peripheral portion 65c that surrounds an outer peripheral surface of the pole piece 64 in a separated manner, a power transmission coil 62 disposed in a gap between the pole piece 64 and the yoke 65 by winding the pole piece 64, and a sealing case 66 made of a non-magnetic material that integrally covers the pole piece 64, the yoke 65, and the power transmission coil 62. In this example, the power transmission coil unit 61 is disposed on a side facing the spray unit 12 along the upper wall 11Mt of the main body casing 11M. With this configuration, the power transmission coil 62 is disposed in a specific region along the inner side (wall surface) of the upper wall 11Mt forming the main body casing 11M, that is, a region 11a (in FIG. 2, the outer diameter of the region 11a is indicated by a double-headed arrow) surrounding the recess 11K1 about the longitudinal axis 11C. The power transmission coil 62 is connected to the control unit 60 by wirings 63a and 63b. The power transmission coil 62 is used to transmit the oscillation output from the control unit 60 to the spray unit 12 with the wireless power transmission system.

The spray unit 12 is mounted with the horn vibrator 40 as a vibration unit and a power reception coil unit 71 to accommodate them in the attachment casing 30 (in particular, the base casing 30M).

As shown in FIG. 2, the horn vibrator 40 is configured by integrally combining a vibration surface 43 arranged horizontally upward, an ultrasonic vibrator 41 arranged at a position separated downward from the vibration surface 43, and a horn 42 arranged between the ultrasonic vibrator 41 and the vibration surface 43. The horn 42 amplifies the vibration of the ultrasonic vibrator 41, and transmits the vibration to the vibration surface 43. In a state where the cover member 31 is attached to the base casing 30M, a gap 43g is present between the sheet 21 including the mesh portion and the vibration surface 43 of the horn vibrator 40. As described later, the first liquid (or the second or the third liquid) in the liquid reservoir 17 is supplied to the gap 43g. The horn vibrator 40 and (a power reception coil 72 of) the power reception coil unit 71 are connected by wirings 73a and 73b.

The power reception coil unit 71 includes a pole piece 74 made of a substantially columnar magnetic body, a power reception coil 72 disposed around the pole piece 74 by winding the pole piece 74, and a sealing case 75 made of a non-magnetic material integrally covering the pole piece 74 and the power reception coil 72. In this example, the power reception coil unit 71 is disposed on a side facing the main body 11 along an inner side of the bottom wall 30Mb of the base casing 30M. With this configuration, the power reception coil 72 is disposed, along the inner side (wall surface) of the bottom wall 30Mb forming the base casing 30M, in a region 12a (in FIG. 2, the outer diameter of the region 12a is indicated by a double-headed arrow), which corresponds to the region 11a of the main body casing 11M where the power transmission coil 62 is disposed.

As a result, in a state where the main body 11 and the spray unit 12 are attached to each other, the power transmission coil 62 and the power reception coil 72 are disposed in the regions 11a and 12a corresponding to each other with the upper wall 11Mt forming the main body casing 11M and the bottom wall 30Mb forming the attachment casing 30 interposed therebetween. Thus, during operation, the oscillation output from the control unit 60 is efficiently transmitted from the main body 11 to the spray unit 12 with the wireless power transmission system via the power transmission coil 62 and the power reception coil 72.

(Operation of Nebulizer)

A user who intends to use the nebulizer 1 attaches any one of the spray units 12-1, 12-2, and 12-3 to the main body 11, and puts the first, second, or third liquid suitable for the spray unit 12 into the liquid reservoir 17 of the attached spray unit 12. This causes the liquid put in the liquid reservoir 17 to be supplied to the gap 43g (see FIG. 2) between the sheet 21 and the vibration surface 43 of the horn vibrator 40. The mouthpiece 80 is attached to the opening 31o of the spray unit 12. Subsequently, as shown in FIG. 5, the user 99 tilts the entire nebulizer 1 toward the front side, brings the mouthpiece 80 close to the mouth, and holds the mouthpiece in the mouth. In this state, the user 99 turns on the power switch 50A provided on the front surface 11Ms of the main body 11.

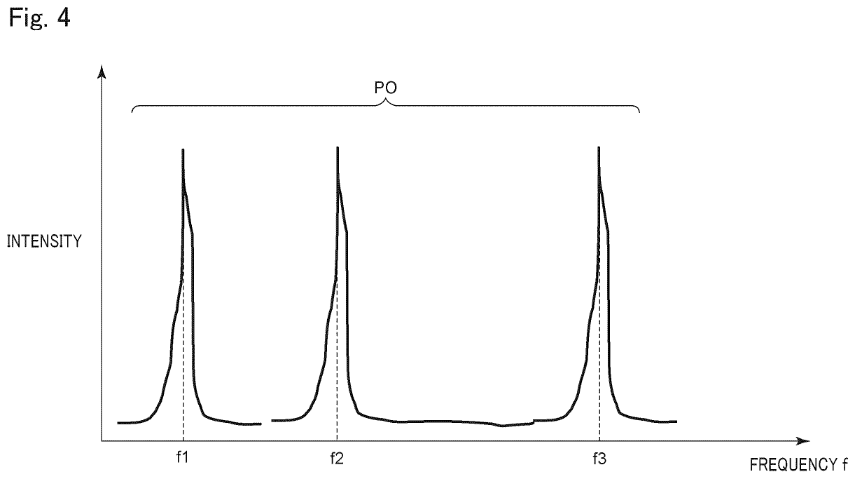

Then, the control unit 60 acts as an oscillation unit and generates an oscillation output PO including a predetermined first frequency component f1, second frequency component f2, and third frequency component f3 different from each other as shown in FIG. 4. In this example, the frequency components are set as f1≈180 kHz, f2≈300 kHz, and f3≈500 kHz in advance to suit the atomization with the horn vibrators 40-1, 40-2, and 40-3 in the spray units 12-1, 12-2, and 12-3. Note that the control unit 60 may act as a search unit to sweep the oscillation frequency f to finely adjust (for example, ±5 kHz) the frequencies of the first frequency component f1, the second frequency component f2, and the third frequency component f3 to a frequency (target frequency) at which the efficiency of the atomization operation by the atomization unit can be increased and stabilized in consideration of characteristic variations of the individual horn vibrators 40 based on the relationship between voltage and current supplied from the main body 11 to the spray unit 12. The oscillation output PO is transmitted from the power transmission coil 62 to the power reception coil 72 by wireless power transmission using magnetic coupling. The oscillation output PO received by the power reception coil 72 is applied to the horn vibrator 40 via the wirings 73a and 73b, and the vibration surface 43 vibrates. This causes the liquid (first, second or third liquid) supplied to the gap 43g between the sheet 21 including the mesh portion and the vibration surface 43 of the horn vibrator 40 to be atomized through the sheet 21 including the mesh portion and ejected as an aerosol 90 through the mouthpiece 80 as shown in FIG. 5.

Here, for example, it is assumed that the spray unit 12 attached to the main body 11 by the user is the spray unit 12-1. In a state where the spray unit 12-1 is attached to the main body 11, during operation, the spray unit 12-1 receives the oscillation output PO including the first frequency component f1, the second frequency component f2, and the third frequency component f3 from the main body 11, and the horn vibrator 40-1 mounted on the spray unit 12-1 atomizes the supplied first liquid using the first frequency component f1. This configuration can appropriately atomize the first liquid. Alternatively, it is assumed that the spray unit 12 attached to the main body 11 by the user is the spray unit 12-2. In a state where the spray unit 12-2 is attached to the main body 11, during operation, the spray unit 12-2 receives the oscillation output PO including the first frequency component f1, the second frequency component f2, and the third frequency component f3 from the main body 11, and the horn vibrator 40-2 mounted on the spray unit 12-2 atomizes the supplied second liquid using the second frequency component f2. This configuration can appropriately atomize the second liquid. Alternatively, it is assumed that the spray unit 12 attached to the main body 11 by the user is the spray unit 12-3. In a state where the spray unit 12-3 is attached to the main body 11, during operation, the spray unit 12-3 receives the oscillation output PO including the first frequency component f1, the second frequency component f2, and the third frequency component f3 from the main body 11, and the horn vibrator 40-3 mounted on the spray unit 12-3 atomizes the supplied third liquid using the third frequency component f3. This configuration can appropriately atomize the third liquid. In this manner, according to the nebulizer 1, the first, second, and third liquids having different target frequencies suitable for atomization can be appropriately atomized and can thus respond to various needs.

In particular, according to the nebulizer 1, the user does not need to change the settings (in particular, the frequency components included in the oscillation output PO) of the control unit 60 as the oscillation unit of the main body 11, and can easily and appropriately atomize the first, second, and third liquids by replacing and attaching the spray units 12-1, 12-2, and 12-3 to the main body 11.

In addition, in the nebulizer 1, since power transmission from the main body 11 to the spray unit 12 attached is performed with a wireless power transmission system, it is not necessary to attach and detach any wiring (or any contact) when the user replaces and attaches the spray units 12-1, 12-2, or 12-3 to the main body 11. Thus, the user can easily perform operation of replacing and attaching the spray units 12-1, 12-2, or 12-3 to the main body 11.

In the nebulizer 1, the main body 11 is protected by the main body casing 11M, and the spray units 12-1, 12-2, and 12-3 as the first, second and third replacement members are protected by the attachment casing 30. In particular, when the main body casing 11M and the attachment casing 30 are configured to be liquid-tight, they are protected from infiltration of liquid (for example, the first to third liquids, washing water, and the like).

In this example, since the nebulizer 1 is configured as a mesh-type nebulizer, the atomization unit can be configured to be small, and thus, the spray units 12-1, 12-2, and 12-3 can be configured to be small. In addition, by downsizing the power supply unit 53 and the control unit 60 (reducing the oscillation output PO), the main body 11 can also be downsized, accordingly. This makes it possible to realize a nebulizer that is small as a whole and excellent in portability.

In the above example, three types of spray units 12 (spray units 12-1, 12-2, and 12-3) are attached to the main body 11 in a replaceable manner, but the present invention is not limited to this configuration. The number of spray units 12 to be attached in a replaceable manner may be four or more, or two. In such a case, the control unit 60 of the main body 11 may generate the oscillation output PO including the frequency components corresponding to all the spray units 12.

### First Modification Example

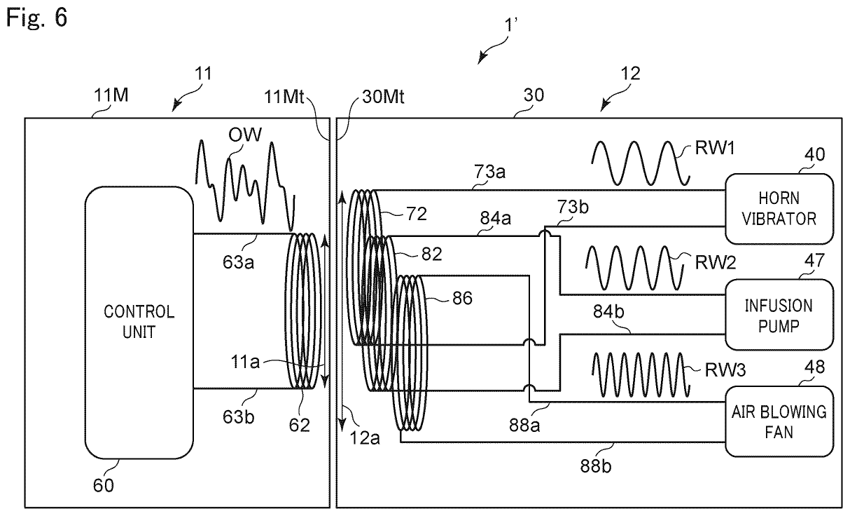

In the nebulizer 1 described above, the spray unit 12 includes only the horn vibrator 40 as an atomization unit (in particular, the vibration unit) as a functional unit that operates in response to the oscillation output PO from the control unit 60, but the present invention is not limited to this configuration. FIG. 6 shows a nebulizer 1′ according to a modification example. The nebulizer 1′ is mounted with, in addition to the horn vibrator 40 as an atomization unit, an infusion pump 47 as a liquid supply unit and an air blowing fan 48 as an air blower unit to accommodate them in the spray unit 12 (at least one of the spray units 12-1, 12-2, and 12-3) as functional units that operate in response to the oscillation output PO from the control unit 60. In FIG. 6, the same components as those in FIGS. 1 to 3 are denoted by the same reference numerals, and redundant description is omitted. The infusion pump 47 is used to supply liquid from the liquid reservoir 17 toward an atomization unit (for example, the gap 43g between the sheet 21 and the vibration surface 43 of the horn vibrator 40). The air blowing fan 48 is used to flow aerosol generated by atomization through a pipe member (such as the mouthpiece 80). The infusion pump 47 and the air blowing fan 48 are configured to respectively operate with predetermined additional frequency components f4 and f5 different from the above-described frequency components f1, f2, and f3. Accordingly, in this example, the control unit 60 generates the oscillation output PO including the additional frequency components f4 and f5 in addition to the first to third frequency components f1, f2, and f3 during operation. For convenience of understanding, a waveform OW of the oscillation output PO that changes over time is schematically shown on the main body 11 side in FIG. 6. A waveform RW1 of the frequency component (f1, f2 or f3) received by the horn vibrator 40 that changes over time, a waveform RW2 of the frequency component f4 received by the infusion pump 47 that changes over time, and a waveform RW3 of the frequency component f5 received by the air blowing fan 48 that changes over time are schematically shown on the spray unit 12 side.

In this example, in the attachment casing 30, a power reception coil 82 for the infusion pump 47 and a power reception coil 86 for the air blowing fan 48 are disposed together in the region 12a where the power reception coil 72 for the horn vibrator 40 is disposed so as to receive the oscillation output PO from the control unit 60. In FIG. 6, for easy understanding, the three power reception coils 72, 82, and 86 are drawn to be shifted from each other, but these power reception coils 72, 82, and 86 may be concentrically wound around the same pole piece 74. The power reception coil 82 and the infusion pump 47 are connected by wirings 84a and 84b. The power reception coil 86 and the air blowing fan 48 are connected by wirings 88a and 88b.

In this nebulizer 1′, the spray unit 12 attached to the main body 11 receives, during operation, the oscillation output PO that includes the additional frequency components f4 and f5 in addition to the frequency components f1, f2, and f3 from the main body 11. The atomization unit (in particular, the horn vibrator 40) of the spray unit 12 operates using the frequency component f1, f2, or f3. Together with this, the functional units (in this example, the infusion pump 47 and the air blowing fan 48) of the spray unit 12 operate with the additional frequency components f4 and f5 different from the frequency components f1, f2, and f3. Thus, even when the attached spray unit 12 includes a functional unit different from the atomization unit, the user can appropriately operate the functional unit without changing the settings (in particular, the frequency components included in the oscillation output PO) of the control unit 60 of the main body 11. Thus, with the operation by the functional unit, it is possible to further respond to various needs.

The control unit 60 may act as a search unit to sweep the oscillation frequency f, and thus to finely adjust (for example, ±5 kHz) the frequencies of the additional frequency components f4 and f5 to frequencies (target frequencies) suitable for the operation of the infusion pump 47 and the air blowing fan 48, respectively, based on the relationship between voltage and current supplied from the main body 11 to the spray unit 12.

### Second Modification Example

In each of the above-described examples, the control unit 60 generates the oscillation output PO including the predetermined frequency components f1, f2, and f3 (or predetermined frequency components f1, f2, f3, f4, and f5) during operation, but the present invention is not limited to this configuration. The control unit 60 may act as a search unit to sweep the oscillation frequency f, for example, in the vicinity of predetermined frequency components f1, f2, and f3, and thus to obtain a target frequency for each of the frequency components f1, f2, and f3 to be supplied to the spray unit 12 attached to the main body 11. Alternatively, the control unit 60 may act as a search unit to sweep the oscillation frequency f in a wider range, and thus to search whether there is a new frequency component (that is, an unexpected frequency component, denoted by reference fx) to be supplied to the spray unit 12 attached to the main body 11. Then, when a new frequency component fx is found, the control unit 60 may act as an oscillation unit to include the new frequency component fx in the oscillation output PO. Thus, even when the user does not particularly change the settings (in particular, the frequency components included in the oscillation output PO) of the control unit 60 as an oscillation unit of the main body 11, a new functional unit (including an atomization unit) to be operated by the new frequency component fx can be operated.

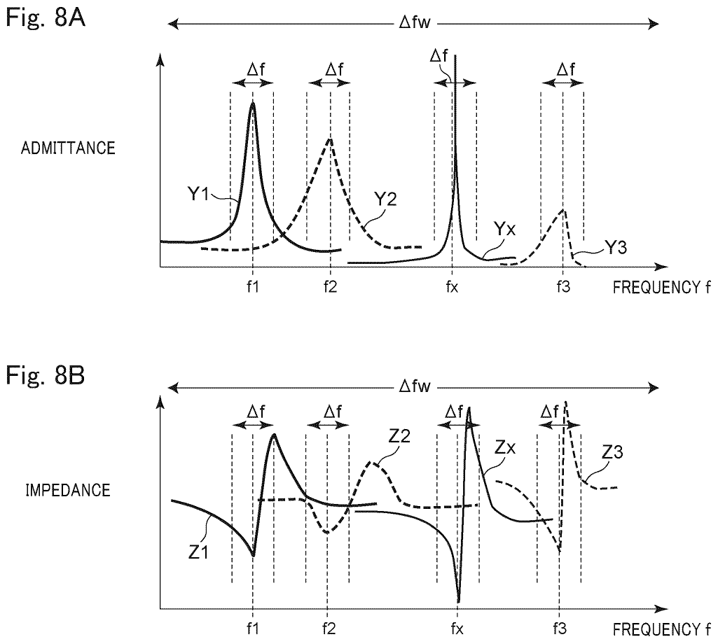

For example, it is assumed that all the horn vibrators 40-1, 40-2, and 40-3 described above are mounted on a certain spray unit 12, and further, a new functional unit (including an atomization unit) (denoted by reference 4x) to be operated by the new frequency component fx is mounted. In this case, when the oscillation frequency f is swept, as shown in FIG. 8A, peaks of the admittances Y1, Y2, and Y3 (or current values) are observed at f1 z 180 kHz, f2 z 300 kHz, and f3 z 500 kHz, respectively, and further, a peak of a new admittance (denoted by reference Yx) is observed in the vicinity of fx. In this example, f2<fx<f3 is assumed, but the present invention is not limited to this configuration. Along with this, as shown in FIG. 8B, steep changes in the impedances Z1, Z2, and Z3 (or voltage values) are observed at f1 z 180 kHz, f2 z 300 kHz, and f3 z 500 kHz, respectively, and further, a steep change in a new impedance (denoted by reference Zx) is observed in the vicinity of fx. Thus, for example, by calculating the voltage/current ratio for each oscillation frequency f as the relationship between voltage and current, the control unit 60 can find the new frequency component fx to be supplied in addition to the predetermined frequency components f1, f2, and f3.

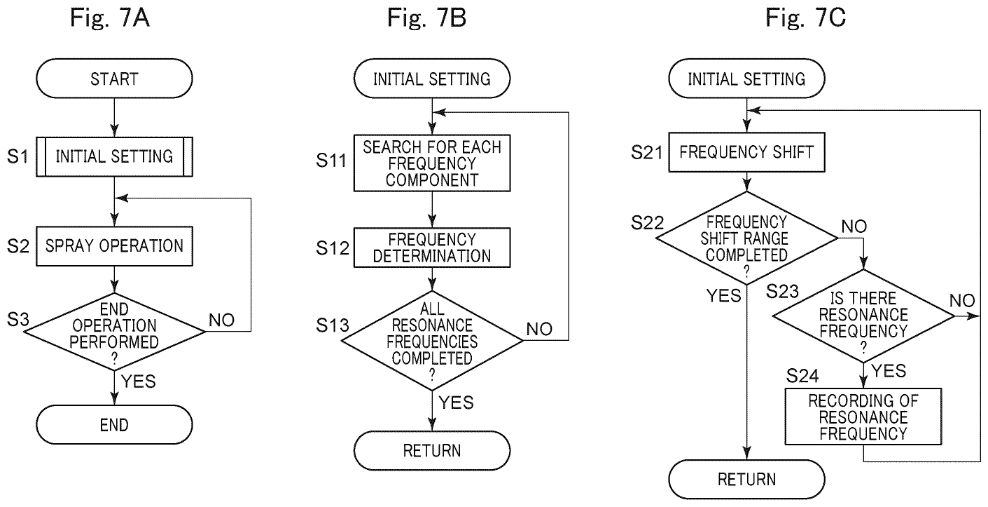

Specifically, as shown in FIG. 7A, when the user turns on the power switch 50A, the control unit 60 first performs an initial setting in step S1.

For example, it is assumed that the initial setting is performed according to the flow shown in FIG. 7B. As described above, it is assumed that f1≈180 kHz, f2≈300 kHz, and f3≈500 kHz. In this flow, the control unit 60 performs a search by sweeping the oscillation frequency f in the vicinity of the frequency components f1, f2, and f3 for each frequency component (in this example, f1, f2, and f3) to be included in the oscillation output PO. Specifically, first, in step S11, as shown in FIGS. 8A and 8B, the oscillation frequency f is swept in the vicinity of the frequency component f1, for example, in the range of Δf=±100 Hz. By doing this, as shown in step S12, the control unit 60 to determine the target frequency (resonance frequency) for the frequency component f1 based on the relationship between voltage and current supplied from the main body 11 to the spray unit 12. Next, unless determination for all target frequencies is completed (NO in step S13), the process returns to step S11, and the oscillation frequency f is swept in the vicinity of the next frequency component f2, for example, in the range of Δf=±100 Hz as shown in FIGS. 8A and 8B. By doing this, as shown in step S12, the control unit 60 to determine the target frequency (resonance frequency) for the frequency component f2. This process is continued until determination for all target frequencies is completed (YES in step S13). In this manner, the target frequencies for the predetermined frequency components f1, f2, and f3 are determined. Then, the process proceeds to step S2 of FIG. 7A described later.

Here, in the flow shown in FIG. 7B, since the oscillation frequency f is only swept in the vicinity of the predetermined frequency components f1, f2, and f3, the initial setting can be completed in a relatively short time. However, when there is a new frequency component fx to be supplied from the main body 11 to the spray unit 12 as described above, the new frequency component fx cannot be found. Thus, for example, the initial setting may be performed according to the flow shown in FIG. 7C.

In the flow shown in FIG. 7C, in step S21, the control unit 60 acts as a search unit to sweep the oscillation frequency f in a wide range (referred to as “frequency shift range”) Δfw from 100 Hz to 2.5 GHz, for example, so as to cover all of the predetermined frequency components f1, f2, and f3 as shown in FIGS. 8A and 8B. Unless the sweep over the entire frequency shift range Δfw is completed (NO in step S22), the control unit 60 continues the sweep, and when there is a frequency component (resonance frequency) to be supplied from the main body 11 to the spray unit 12 (YES in step S23), the control unit 60 records the resonance frequency as a target frequency (step S24). When the sweep of the entire frequency shift range Δfw is completed (YES in step S22), the control unit 60 proceeds to step S2 in FIG. 7A described later.

First, when the predetermined frequency components f1, f2, and f3 cannot be detected at all (or any of them cannot be detected) after the initial setting is attempted in the flow shown in FIG. 7B, the initial setting may be performed according to the flow shown in FIG. 7C.

Here, for example, it is assumed that only f1 among the predetermined frequency components f1, f2, and f3 and a new frequency component fx are found by initial setting according to the flow shown in FIG. 7C. In other words, it is assumed that f2 and f3 among the predetermined frequency components f1, f2, and f3 are proved to be frequency components that do not need to be supplied. Then, in this example, the control unit 60 acts as an oscillation unit to exclude the frequency components f2 and f3 that do not need to be supplied from the oscillation output PO. With this configuration, even when the user does not particularly change the settings (in particular, the frequency components included in the oscillation output PO) of the control unit 60 of the main body 11, the supply of the frequency component (in this example, f2 and f3) that does not need to be supplied can be stopped, and power saving can be performed.

In step S2 of FIG. 7A, the control unit 60 acts as an oscillation unit to generate the oscillation output PO including (only) the frequency components f1 and fx set as the target frequencies in the above example. The oscillation output PO is transmitted from the power transmission coil 62 to the power reception coil 72 for the atomization unit (in this example, the atomization unit constituted by the horn vibrator 40-1 and the mesh member 20-1) and the power reception coil (not shown) for the functional unit 4x by wireless power transmission using magnetic coupling. With this configuration, along with the operation of the functional unit 4x, a spraying operation, that is, atomization of liquid (in this example, the first liquid) by the atomization unit is performed. In this example, the spray operation is continued unless the user performs an end operation (turning off the power switch 50A) (NO in step S3). When the user performs the end operation (YES in step S3), the spray operation ends.

In the above example, only f1 among the predetermined frequency components f1, f2, and f3 and the new frequency component fx are found by the initial setting according to the flow shown in FIG. 7C, but the present invention is not limited to this configuration. For example, only the predetermined frequency component f1 may be found. In this case, in step S24 of FIG. 7C, the control unit 60 records only the target frequency (resonance frequency) for the frequency component f1. Further, in step S2 of FIG. 7A, the control unit 60 acts as an oscillation unit to generate the oscillation output PO including only the frequency component f1 set as the target frequency in the above example.

In the above example, the oscillation frequency f is swept in the frequency shift range Δfw from 100 Hz to 2.5 GHz in the initial setting according to the flow shown in FIG. 7C, but the present invention is not limited to this configuration. The search may be started not from 100 Hz but from, for example, 250 kHz.

In the above-described embodiment, the system for transmitting the oscillation output PO from the main body 11 to the spray unit 12 is the wireless power transmission system, but the present invention is not limited to this configuration. The system for transmitting the oscillation output PO from the main body 11 to the spray unit 12 may be a wired power transmission system. In this case, it is desirable to provide a pair of contacts at a pair of places separated from each other on the outer side (upper side) of the upper wall 11Mt of the main body casing 11M, and to provide a pair of contacts at a pair of places separated from each other on the outer side (lower side) of the bottom wall 30Mb of the spray unit 12 corresponding to the pair of places. With this configuration, when the user attaches the spray unit 12 to the main body 11, the pair of contacts come into contact with each other correspondingly. As a result, the oscillation output PO can be transmitted from the main body 11 to the spray unit 12 with a simple configuration (a wired power transmission system with contacts).

In the above-described embodiment, the main body 11 (and the spray unit 12) has an oval planar shape, but the present invention is not limited to this configuration. The planar shape of the main body 11 (and the spray unit 12) may be an ellipse, a circle, a rounded rectangle (a rectangle with rounded corners), or the like.

In the above-described embodiment, a mesh-type nebulizer has been described, but the present invention is not limited to thereto. The present invention may also be applied to an ultrasonic nebulizer having a so-called two-tank structure (that is, a nebulizer of a type in which a chemical liquid tank is immersed in a cooling water tank facing an ultrasonic vibrator, an ultrasonic vibration energy generated from the ultrasonic vibrator concentrates on a surface of a chemical liquid through cooling water, and the chemical liquid is atomized by an action of vibration (cavitation effect)).

As described above, a nebulizer of the present disclosure is a nebulizer that atomizes and ejects a liquid, the nebulizer comprising:
a main body being mounted with a power supply unit and an oscillation unit, the oscillation unit receiving power supply from the power supply unit and generating an oscillation output including a predetermined first frequency component and a predetermined second frequency component different from each other;
a first replacement member being mounted with an atomization unit configured to atomize, using the first frequency component, a first liquid that is supplied; and
a second replacement member being mounted with an atomization unit configured to atomize, using the second frequency component, a second liquid that is supplied,
wherein
the first replacement member and/or the second replacement member include a functional unit configured to operate with a predetermined additional frequency component different from the first and second frequency components,
the oscillation unit of the main body generates the oscillation output including the additional frequency component in addition to the first and second frequency components, and
the first replacement member or the second replacement member is attached to the main body in a replaceable manner, and a replacement member attached to the main body receives the oscillation output including the first frequency component, the second frequency component, and the additional frequency component from the main body.

In the present specification, for the “power supply unit”, a battery may be used, or a unit that converts a commercial power supply may be used.

The “first liquid” and the “second liquid” refer to, for example, chemical liquids having different viscosities from each other.

The “first frequency component” and the “second frequency component” have frequencies (target frequencies) suitable for atomization of the “first liquid” and the “second liquid”, respectively.

The “first replacement member” and the “second replacement member” typically include ultrasonic vibrators for atomization operations, respectively. The “first replacement member” may further include a liquid supply unit that supplies the first liquid to its own atomization unit, and the “second replacement member” may further include a liquid supply unit that supplies the second liquid to its own atomization unit.

The “functional unit” means an element that operates by receiving the oscillation output from the main body (to this extent, the “functional unit” is a concept including the atomization unit as well). The “functional unit” may be, for example, a liquid supply unit (including an infusion pump, for example) that supplies a liquid to the atomization unit, or an air blower unit (for example, an air blowing fan) that assists ejection of atomized liquid.

The “attached replacement member” refers to the first replacement member or the second replacement member attached to the main body.

The system for transmitting the “oscillation output” from the main body to the first replacement member or the second replacement member may be a wireless power transmission system or a wired power transmission system.

In the nebulizer of this disclosure, the oscillation unit of the main body generates the oscillation output including the additional frequency component in addition to the first and second frequency components during operation. The attached replacement member (the first replacement member or the second replacement member) receives the oscillation output including the additional frequency component in addition to the first and second frequency components from the main body. For example, it is assumed that a user attaches the first replacement member to the main body. In a state where the first replacement member is attached to the main body, during operation, the first replacement member receives the oscillation output including the first frequency component, the second frequency component, and the additional frequency component from the main body. This causes the atomization unit mounted on the first replacement member to atomize, using the first frequency component, a first liquid that is supplied. This configuration can appropriately atomize the first liquid. Also, the functional unit mounted on the first replacement member operates with the additional frequency component different from the first and second frequency components. Alternatively, it is assumed that the user attaches the second replacement member to the main body. In a state where the second replacement member is attached to the main body, during operation, the second replacement member receives the oscillation output including the first frequency component and the second frequency component from the main body. This causes the atomization unit mounted on the second replacement member to atomize, using the second frequency component, a second liquid that is supplied. This configuration can appropriately atomize the second liquid. Also, the functional unit mounted on the second replacement member operates with the additional frequency component different from the first and second frequency components. In this manner, according to the nebulizer, the first and second liquids having different target frequencies suitable for atomization can be appropriately atomized, and the functional unit mounted on the attached replacement member can be operated. Thus, it is possible to respond to various needs.

In particular, according to the nebulizer, the user does not need to change the settings (in particular, the frequency components included in the oscillation output) of the oscillation unit of the main body, and can appropriately atomize each of the first and second liquids simply by replacing and attaching the first replacement member or the second replacement member to the main body. Furthermore, even when the attached replacement member includes a functional unit different from the atomization unit, an operation by the functional unit can be performed without the user changing the settings (in particular, the frequency components included in the oscillation output) of the oscillation unit of the main body.

In the nebulizer according to one embodiment,
the main body includes a power transmission coil for transmitting the oscillation output on a side facing the replacement member that is attached,
each of the first replacement member and the second replacement member includes a power reception coil for receiving the oscillation output on a side facing the main body, and
the replacement member that is attached is configured to receive the oscillation output from the main body with a wireless power transmission system using magnetic coupling between the power transmission coil and the power reception coil.

The “wireless power transmission system using magnetic coupling” widely includes electromagnetic induction systems and magnetic field resonance systems.

In the nebulizer according to this embodiment, since the power transmission from the main body to the attached replacement member is performed with the wireless power transmission system, it is not necessary to attach and detach a wiring (or a contact) when the user replaces and attaches the first replacement member or the second replacement member to the main body. Thus, the user can easily perform an operation of replacing and attaching the first replacement member or the second replacement member to the main body.

In the nebulizer according to one embodiment,
the main body includes a main body casing that accommodates the power supply unit and the oscillation unit, and the power transmission coil is disposed in a specific region along an inner side of a wall surface forming the main body casing, and
each of the first replacement member and the second replacement member includes an attachment casing that accommodates the atomization unit, and the power reception coil is disposed in a region corresponding to the specific region of the main body casing along an inner side of a wall surface forming the attachment casing.

In the nebulizer according to this embodiment, between the main body and the attached replacement member (the first replacement member or the second replacement member), the power transmission coil and the power reception coil are disposed in regions corresponding to each other with a wall surface forming the main body casing and a wall surface forming the attachment casing interposed between the coils. Thus, wireless power transmission between the power transmission coil and the power reception coil is efficiently performed. In the nebulizer according to this embodiment, the main body is protected by the main body casing, and the first and second replacement members are protected by the attachment casing. In particular, when the main body casing and the attachment casing are each configured to be liquid-tight, the main body casing and the attachment casing are protected from intrusion of liquid (for example, the first and second liquids, washing water, and the like).

In the nebulizer according to one embodiment,
the first replacement member and/or the second replacement member including the functional unit include, as the power reception coil, a first power reception coil for the atomization unit and a second power reception coil for the functional unit, and
the first and second power reception coils are concentrically wound around a same pole piece.

In the nebulizer according to one embodiment,
the main body includes a search unit, and
the search unit sweeps an oscillation frequency generated by the oscillation unit in a certain frequency range during operation, and thus obtains a target frequency for each of the frequency components based on a relationship between voltage and current supplied from the main body to the replacement member that is attached.

The “target frequency” refers to a frequency to be targeted for each frequency component. The “target frequency” for the first and second frequency components corresponds to frequencies suitable for atomization of the first and second liquids with the atomization units of the first and second replacement members, respectively. Here, the “suitable” frequency refers to a frequency at which the efficiency of the atomization operation of the atomization unit can be increased and stabilized in consideration of, for example, characteristic variations of individual ultrasonic vibrators included in the atomization units. In the same manner, the “target frequency” for the additional frequency component corresponds to a frequency suitable for the operation of the functional unit.

In the nebulizer according to this embodiment, the search unit sweeps an oscillation frequency generated by the oscillation unit in a certain frequency range during operation and obtains a target frequency for each of the frequency components based on the relationship between voltage and current supplied from the main body to the attached replacement member. As a result, the oscillation unit can set the frequency of each of the frequency components to the target frequency. Thus, it is possible to increase and stabilize the efficiency of the atomization operation of the atomization units of the first and second replacement members. In addition, when the first replacement member and/or the second replacement member includes the functional unit, the functional unit can be appropriately operated.

In the nebulizer according to one embodiment,
the search unit sweeps the oscillation frequency generated by the oscillation unit in the certain frequency range during operation, and thus searches whether there is a new frequency component to be supplied to the replacement member that is attached based on the relationship between voltage and current, and
when the new frequency component is found, the oscillation unit includes the new frequency component in the oscillation output.

The “new frequency component” is different from the previously described predetermined frequency component (the first and second frequency components and the additional frequency component) and refers to an unexpected frequency component. The functional unit to be operated by the new frequency component may be a new atomization unit different from the above atomization units.

In the nebulizer according to this embodiment, the search unit sweeps the oscillation frequency generated by the oscillation unit in the certain frequency range during operation, and thus searches whether there is a new frequency component to be supplied to the attached replacement member based on the relationship between voltage and current. When the new frequency component is found, the oscillation unit includes the new frequency component in the oscillation output. Thus, even when the user does not particularly change the settings (in particular, the frequency components included in the oscillation output) of the oscillation unit of the main body, a new functional unit (including an atomization unit) to be operated by the new frequency component can be operated.

In the nebulizer according to one embodiment,
the search unit sweeps the oscillation frequency generated by the oscillation unit in the certain frequency range during operation, and thus searches whether there is a frequency component that does not need to be supplied to the replacement member that is attached among the frequency components based on the relationship between voltage and current, and
when it is found that there is the frequency component that does not need to be supplied, the oscillation unit excludes the frequency component that does not need to be supplied from the oscillation output.

In the nebulizer according to this embodiment, the search unit sweeps the oscillation frequency generated by the oscillation unit in the certain frequency range during operation, and thus searches whether there is a frequency component that does not need to be supplied to the attached replacement member among the frequency components based on the relationship between voltage and current. When it is found that there is the frequency component that does not need to be supplied, the oscillation unit excludes the frequency component that does not need to be supplied from the oscillation output. Thus, even when the user does not particularly change the settings (in particular, the frequency components included in the oscillation output) of the oscillation unit of the main body, any supply of the frequency component that does not need to be supplied can be stopped, and power saving can be achieved.

In the nebulizer according to one embodiment,
the atomization unit of the first replacement member includes a vibration unit including a vibration surface and operating using the first frequency component and a mesh member including a mesh portion disposed facing the vibration surface, the atomization unit of the first replacement member being configured to atomize, through the mesh portion, the first liquid supplied between the vibration surface and the mesh portion during operation, and
the atomization unit of the second replacement member includes a vibration unit including a vibration surface and operating using the second frequency component and a mesh member including a mesh portion disposed facing the vibration surface, the atomization unit of the second replacement member being configured to atomize, through the mesh portion, the second liquid supplied between the vibration surface and the mesh portion during operation.

The “mesh portion” means an element that has a plurality of through holes formed in a sheet or a plate material and allows a liquid to pass through these through holes to be atomized.

In the nebulizer according to this embodiment, the atomization unit of the first replacement member includes a vibration unit having a vibration surface and operating using the first frequency component, and a mesh member having a mesh portion disposed facing the vibration surface. The atomization unit atomizes, through the mesh portion, the first liquid supplied between the vibration surface and the mesh portion during operation. The atomization unit of the second replacement member includes a vibration unit having a vibration surface and operating using the second frequency component, and a mesh member having a mesh portion disposed facing the vibration surface. The atomization unit atomizes, through the mesh portion, the second liquid supplied between the vibration surface and the mesh portion during operation. That is, the nebulizer is configured as a mesh-type nebulizer, the atomization unit can be configured to be small, and thus, the first and second replacement members can be configured to be small. In addition, by downsizing the power supply unit and the oscillation unit (reducing the oscillation output), the main body can also be downsized, accordingly. This makes it possible to realize a nebulizer that is small as a whole and excellent in portability.

As is apparent from the above, according to the nebulizer of the present disclosure, liquids having different target frequencies suitable for atomization can be appropriately atomized, and thus, it is possible to respond to various needs.

The above embodiments are illustrative, and are modifiable in a variety of ways without departing from the scope of this invention. It is to be noted that the various embodiments described above can be appreciated individually within each embodiment, but the embodiments can be combined together. It is also to be noted that the various features in different embodiments can be appreciated individually by its own, but the features in different embodiments can be combined.

1. A nebulizer that atomizes and ejects a liquid, the nebulizer comprising:
a main body being mounted with a power supply unit and an oscillation unit, the oscillation unit receiving power supply from the power supply unit and generating an oscillation output including a predetermined first frequency component and a predetermined second frequency component different from each other;
a first replacement member being mounted with an atomization unit configured to atomize, using the first frequency component, a first liquid that is supplied; and
a second replacement member being mounted with an atomization unit configured to atomize, using the second frequency component, a second liquid that is supplied,
wherein
the first replacement member and/or the second replacement member include a functional unit configured to operate with a predetermined additional frequency component different from the first and second frequency components,
the oscillation unit of the main body generates the oscillation output including the additional frequency component in addition to the first and second frequency components, and
the first replacement member or the second replacement member is attached to the main body in a replaceable manner, and a replacement member attached to the main body receives the oscillation output including the first frequency component, the second frequency component, and the additional frequency component from the main body.

2. The nebulizer according to claim 1, wherein
the main body includes a power transmission coil for transmitting the oscillation output on a side facing the replacement member that is attached,
each of the first replacement member and the second replacement member includes a power reception coil for receiving the oscillation output on a side facing the main body, and
the replacement member that is attached is configured to receive the oscillation output from the main body with a wireless power transmission system using magnetic coupling between the power transmission coil and the power reception coil.

3. The nebulizer according to claim 2, wherein
the main body includes a main body casing that accommodates the power supply unit and the oscillation unit, and the power transmission coil is disposed in a specific region along an inner side of a wall surface forming the main body casing, and
each of the first replacement member and the second replacement member includes an attachment casing that accommodates the atomization unit, and the power reception coil is disposed in a region corresponding to the specific region of the main body casing along an inner side of a wall surface forming the attachment casing.

4. The nebulizer according to claim 2, wherein
the first replacement member and/or the second replacement member including the functional unit include, as the power reception coil, a first power reception coil for the atomization unit and a second power reception coil for the functional unit, and
the first and second power reception coils are concentrically wound around a same pole piece.

5. The nebulizer according to claim 1, wherein
the main body includes a search unit, and
the search unit sweeps an oscillation frequency generated by the oscillation unit in a certain frequency range during operation, and thus obtains a target frequency for each of the frequency components based on a relationship between voltage and current supplied from the main body to the replacement member that is attached.

6. The nebulizer according to claim 5, wherein
the search unit sweeps the oscillation frequency generated by the oscillation unit in the certain frequency range during operation, and thus searches whether there is a new frequency component to be supplied to the replacement member that is attached based on the relationship between voltage and current, and
when the new frequency component is found, the oscillation unit includes the new frequency component in the oscillation output.

7. The nebulizer according to claim 5, wherein
the search unit sweeps the oscillation frequency generated by the oscillation unit in the certain frequency range during operation, and thus searches whether there is a frequency component that does not need to be supplied to the replacement member that is attached among the frequency components based on the relationship between voltage and current, and
when it is found that there is the frequency component that does not need to be supplied, the oscillation unit excludes the frequency component that does not need to be supplied from the oscillation output.

8. The nebulizer according to claim 1, wherein
the atomization unit of the first replacement member includes a vibration unit including a vibration surface and operating using the first frequency component and a mesh member including a mesh portion disposed facing the vibration surface, the atomization unit of the first replacement member being configured to atomize, through the mesh portion, the first liquid supplied between the vibration surface and the mesh portion during operation, and
the atomization unit of the second replacement member includes a vibration unit including a vibration surface and operating using the second frequency component and a mesh member including a mesh portion disposed facing the vibration surface, the atomization unit of the second replacement member being configured to atomize, through the mesh portion, the second liquid supplied between the vibration surface and the mesh portion during operation.
